(19) United States
(12) Patent Application Publication
(10) Pub. No.: US 2006/0258609 A1
(43) Pub. Date: Nov. 16, 2006

(54) Method for inhibition of lipogenesis by regulating PRP19 expression

(75) Inventors: Si-Young Cho, Yongin-si (KR); Eui-Seok Shin, Yongin-si (KR); Pil-Joon Park, Seoul (KR); Dong-Wook Shin, Seoul (KR); Hui-Kyoung Chang, Yongin-si (KR); Dae-Gun Kim, Seoul (KR); Hyoung-Ho Lee, Yongin-si (KR); Jeong-Ho Lee, Yongin-si (KR); Tae-Ryong Lee, Suwon-si (KR); Ih-Seop Chang, Yongin-si (KR)

Correspondence Address: SUGHRUE MION, PLLC, 2100 PENNSYLVANIA AVENUE, N.W., SUITE 800, WASHINGTON, DC, 20037, US

(73) Assignee: AMOREPACIFIC CORPORATION

(21) Appl. No.: 11/430,131

(22) Filed: May 9, 2006

Related U.S. Application Data
(60) Provisional application No. 60/682,372, filed on May 19, 2005.

(30) Foreign Application Priority Data
May 10, 2005 (KR) 2005-0038693

(51) Int. Cl. A61K 48/00; C12N 15/09

(52) U.S. Cl. 514/44; 435/455

(57) ABSTRACT

The downregulation of PRP 19 (precursor RNA processing 19) protein expression results in effectively reducing the expression of SCD1 (stearyl CoA desaturase-1), the key lipogenic enzyme, and its down-stream triacylglycerol synthesis enzymes, DGAT-1 (diacyglycerol acyltransferase-1) and GPAT (glycerol-phosphate acyltransferase), as well as the intracellular content of neutral lipids, the major target for treating obesity.

### FIELD OF THE INVENTION

The present invention relates to a composition for the inhibition of lipogenesis comprising a regulator of PRP19 (precursor RNA processing 19) protein expression as an active ingredient, a method for inhibiting lipogenesis by regulating PRP19 protein expression, and a method for screening an inhibitor candidate of lipogenesis by analyzing PRP19 gene or its protein expression.

### BACKGROUND OF THE INVENTION

PRP19 (precursor RNA processing 19) protein is a 54 kDa protein containing six WD (tryptophan/aspartic acid) repeat domains, and it has been known to participate in the DNA recombination and repair. However, there has never been reported that PRP19 protein is involved in the lipid metabolism or lipid droplet biogenesis.

Most organisms store surplus nutrition remaining after metabolism in lipid droplets of adipocytes in the form of neutral lipid, mainly triacylglycerol (TAG). The lipid droplet is a subcellular organelle composed of phospholipids and lipid droplet associated proteins, and deposits or transports TAQ diacylglycerol (DAG) and cholesteryl ester. It has been established that several lipid metabolic diseases, e.g., fatty liver, obesity, arteriosclerosis and diabetes are induced by a disorder of lipid droplet biogenesis, and especially, diabetes is caused by the abnormal accumulation of neutral lipids, which results from lipid droplet growing more than twice in size.

The lipid droplet associated proteins can be classified according to the cell type, differentiation stage, and deposition or transportation mode. The representative examples thereof include apolipoprotein B which forms part of very-low density lipoprotein (VLDL), adipocyte differentiation-related protein (ADRP) which surrounds fatty clods of adipocytes, and perilipin. Most of such proteins are expressed in adipocyte-specific manner, and play an important role in carrying TAG to lipid droplets or protecting lipid droplets from lipolysis by lipases.

Therefore, in order to prevent or treat the lipid metabolic diseases, there have been numerous attempts to develop a method for regulating lipolysis or lipid accumulation by modifying the expression or activity of such lipid droplet-associated proteins (Murphy et al., Prog. Lipid Res. (2001), 40, 325-438). However, the essential mechanism of the lipid droplet biogenesis or regulation still remains unresolved.

The present inventors have therefore endeavored to develop a method for effectively inhibiting lipogenesis, and have found that: PRP19 protein is localized within lipid droplets of adipocyte and abundantly expressed in adipose tissue-specific manner; its expression is regulated depending on adipocyte differetiation stages; and the downregulation of PRP19 protein expression dramatically reduces lipid droplet biogenesis and intracellular neutral lipid content.

### SUMMARY OF THE INVENTION

Accordingly, it is a primary object of the present invention to provide a pharmaceutical composition for the inhibition of lipogenesis which can effectively suppress intracelluar lipid droplet biogenesis.

It is another object of the present invention to provide a method for effectively inhibiting lipogenesis.

It is a further object of the present invention to provide an efficient method for screening candidates for lipogenesis inhibitors.

In accordance with one aspect of the present invention, there is provided a pharmaceutical composition for the inhibition of lipogenesis comprising a regulator of PRP19 protein expression as an active ingredient and a pharmaceutically acceptable carrier.

In accordance with another aspect of the present invention, there is provided a method for the inhibition of lipogenesis comprising the step of regulating PRP19 protein expression in a subject in need of lipogenesis inhibition.

In accordance with a further aspect of the present invention, there is provided a method for screening a viable lipogenesis inhibitor comprising the steps of treating a subject with candidate inhibitors and analyzing PRP19 gene or RPR19 protein expression in the subject.

### BRIEF DESCRIPTION OF THE DRAWINGS

The above and other objects and features of the present invention will become apparent from the following description of the invention, when taken in conjunction with the accompanying drawings, which respectively show:

FIG. 1: PRP19 protein expression levels in cells harvested before, and at 2, 4 and 8 days after inducing of the differentiation of 3T3-L1, a mouse embryonic fibroblast-preadipocyte cell line, determined by western blot analyses;

FIG. 2: Immunofluorescence assay results for determining the intracellular localization of expressed PRP1 9 protein in differentiated adipocytes;

FIG. 3: Cleavage map of pSi-PRP19 vector expressing PRP19 siRNA of the present invention;

FIG. 4: Western blot analysis results showing PRP19 protein expression levels in a cell line expressing PRP19 siRNA and a control cell line, respectively;

FIG. 5: Microphotographs for observing lipid droplet biogenesis in a cell line expressing RPR19 siRNA and a control cell line, respectively;

FIG. 6: High performance thin layer chromatography (HPTLC) results showing the amounts of neutral lipids in a cell line expressing PRP19 siRNA and a control cell line, respectively;

FIG. 7: Western blot analysis results obtained for the protein expression of FAS, perilipin, PPAR-γ, C/EBP-α, SCD1 and β-actin in the cell line expressing PRP19 siRNA and the control cell line, obtained in Example 3, respectively; and

FIG. 8: Real-time quantitative RT-PCR analysis results obtained for the mRNA expression of aP2, SREBP-1c, DGAT-1 and GPAT in the cell line expressing PRP19 siRNA and the control cell line, obtained in Example 3, respectively.

### DETAILED DESCRIPTION OF THE INVENTION

The term “PRP19 siRNA” is siRNA capable of mediating RNA interference (RNAi) against the PRP19 gene expression.

The present inventors have discovered that PRP19 protein is expressed in adipose tissue-spectific manner and abundantly localized in lipid droplets of differetiated adipocytes.

The pharmaceutical composition of the present invention is characterized by comprising a regulator that effectively downregulates PRP19 protein expression as an active ingredient. Representative examples of the regulator include antisense RNAs, interfering RNAs (iRNAs) and small interfering RNAs (siRNAs) capable of mediating RNA interference (RNAi) against PRP19 gene expression, and expression vectors thereof, which can be used for downregulating PRP19 protein expression at its mRNA level; transcription inhibitors of PRP19 gene; translation inhibitors of transcribed PRP19 mRNA; and inhibitors of PRP19 protein localization. Among them, PRP19 siRNA and its expression vector are preferable because they can specifically and potently downregulate PRP19 gene expression even when a small amount is applied. More preferably, the regulator is siRNA having the nucleotide sequence of SEQ ID NO: 1 and its expression vector.

The composition of the present invention may further comprise pharmaceutically acceptable carriers, excipients or additives, and preferably, the inventive composition is of the form of an injection formulation for gene therapy.

The inventive composition may be systemically or locally administered to a subject in need of lipogenesis inhibition. A suitable single dose of the active ingredient of the inventive composition for administration to a human (of approximately 70 kg body weight) may be about from 0.001 ng to 1000 μg, but it should be understood that the dose should be determined in light of various relevant factors including the condition to be treated, the route of administration, the age and weight of the patient, and the severity of the patient's symptoms; and, therefore, the dosage suggested above should not be construed to limit the scope of the invention in anyway.

Further, the present invention provides a method for the inhibition of lipogenesis comprising the step of regulating PRP19 protein expression in a subject in need of lipogenesis inhibition. The subject may be a mammal such as a human.

In the method of the present invention, PRP19 protein expression may be regulated by targeting PRP19 gene on the chromosomal DNA though the conventional gene knock-out method; targeting PRP19 mRNA using antisense RNA, iRNA and siRNA capable of mediating RNA interference (RNAi) against PRP19 gene expression, and an expression vector which can introduce one of these RNAs into cells; suppressing the transcription of PRP19 gene; suppressing the translation of transcribed PRP19 mRNA; or inhibiting the intracellular localization of PRP19 protein. The methods mentioned above should not be construed to limit the scope of the invention.

In accordance with the inventive method, it is possible to effectively downregulate the expression of SCD1 (stearyl CoA desaturase-1), which is known as the key lipogenic enzyme, as well as its down-stream triacylglycerol synthesis enzymes, DGAT-1 (diacyglycerol acyltransferase-1) and GPAT (glycerol-phosphate acyltransferase), and it is also possible to reduce the intracellular content of neutral lipids, the major targets for curing obesity, by 70%. Therefore, the inventive method may be beneficially used for preventing or treating lipid metabolic diseases including fatty liver, obesity, arteriosclerosis and diabetes.

Furthermore, the present invention encompasses, within its scope, a method for screening for a viable lipogenesis inhibitor by treating a subject with candidate inhibitors and analyzing PRP19 gene or its protein expression. The inventive method may comprise the steps of treating cell lines (e.g., differentiated 3T3-L1), tissues, or test animals which express PRP19 protein with candidate inhibitors, and analyzing the changes in PRP19 gene or its protein expression levels by using PRP19 gene or a fragment thereof, or an antibody specific for PRP19 protein.

In the inventive method, the expression level of PRP19 gene or PRP19 protein may be analyzed using one of the known methods used for detecting gene or protein expression level. For example, PRP19 gene expression may be analyzed by RT-PCR, or blot analysis such as northern blot using PRP19 gene or a fragment thereof as a probe, and PRP19 protein expression, by way of conducting enzyme-linked immunosorbent assay (ELISA), radioimmunoassay (RIA), sandwich assay, western blot, immunoblot or immunohistochemical staining, using antibodies against PRP 19 protein.

The following Examples are intended to further illustrate the present invention without limiting its scope.

### EXAMPLE 1

### The Expression Level of PRP19 Protein by the Differentiation of Adipocyte

Step 1) Cell Culture and Induction of Adipogenic Differentiation

Cells of mouse undifferentiated adipocytic cell lines, 3T3-L1 (ATCC No. CL-173) were maintained in Dulbecco's modified Eagle medium (DMEM, Gibco CA. 1210-0038) supplemented with 10% goat serum under the condition of 37° C. and 10% CO₂ until 70% confluency. For adipogenic differentiation, 3T3-L1 cells were cultured successively in: DMEM supplemented with 10% FBS, 0.5 mM 3-isobutyl-1-methylxanthine (Sigma), 1 μM dexamethasone (Sigma) and 167 nM insuline (Novo-Nordisk) for 48 hours; DMEM supplemented with 10% FBS and 167 nM insuline for 48 hours; and DMEM supplemented with 10% FBS for 48 hours, to obtain differetiated adipocytes.

Step 2) Analysis for the Expression Level of PRP19 Protein

While 3T3-L1 cells were undergoing adipogenic differentiation for 8 days as described in Step 1), a part of the cells were harvested at intervals of 2 days.

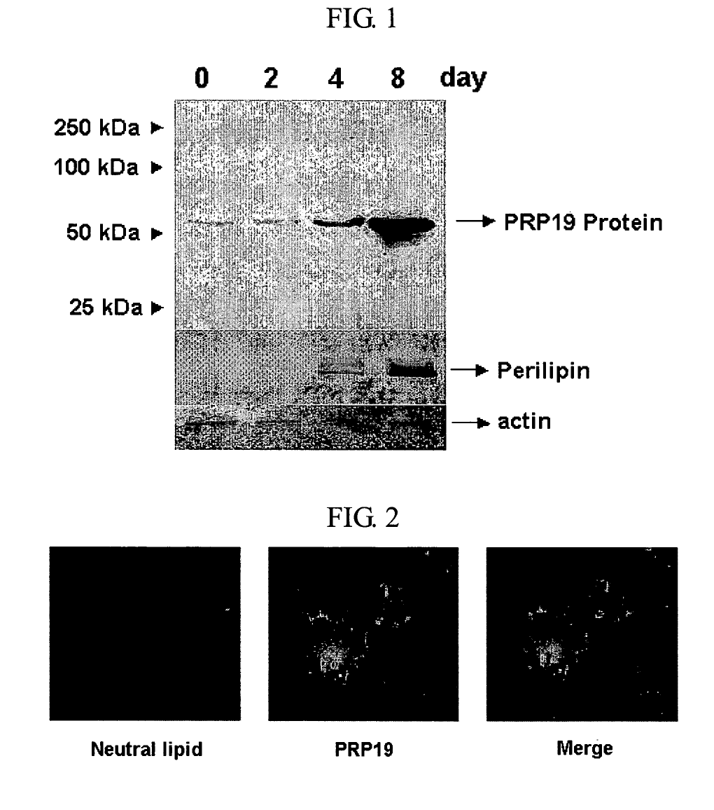

Each harvested cell sample was treated with 1 ml of RIPA solution (1×PBS, 1% Nonidet P-40, 0.5% sodium deoxylate and 0.1% SDS), incubated on ice for 30 min, and centrifuged at 15,000×g, 4° C. for 10 min to obtain a supernatant. After determining the protein content of the supernatant, 40 μg of the protein from the supernatant was loaded on 8% SDS-PAGE gel, and transferred to PDF membrane (BioRad) at 50 V for 12 hours. The transferred membrane was blocked with 5% skim milk solution for 1 hour, and incubated for 1 hour with anti-PRP19 polyclonal antibody (Lab Frontier), anti-perilipin monoclonal antibody (Research Diagnostics), or anti-β-actin monoclonal antibody (Research Diagnostics) as a primary antibody, and with HRP (horse radish peroxidase)-conjugated anti-rabbit IgG (Amersham) as a secondary antibody. The bound antibody was visualized by the chemiluminescent method using ELC kit (enhanced chemiluminescence kit, Amersham), and the result is shown in FIG. 1.

As shown in FIG. 1, the expression of PRP19 protein dramatically increased 4 days after the induction of the adipogenic differentiation, similarly to perilipin, a representative lipid droplet-associated protein.

### EXAMPLE 2

### Intracellular Localization of PRP19 Protein in Adipocyte

In order to study the intracellular localization of PRP19, an immunofluorescence assay was conducted as follows.

The differentiated adipocytes obtained in Step 1) of Example 1 were fixed with 3.7% formaldehyde and washed with PBS 3 times. The fixed cells were blocked with PBS containing 0.1% triton X-100 and 10% FBS for 20 min, and incubated with anti-PRP19 primary antibody and FITC conjugated anti-rabbit IgG secondary antibody (Amersham), followed by adding 0.2% Sudan III to stain neutral lipids. The stained cells were mounted with an anti-fade solution (Molecular Probes) and observed through microscopy. The results are shown in FIG. 2.

As the result in FIG. 2 shows, mouse RPR19 protein was heavily localized on the surface of lipid droplets surrounding neutral lipids, similarly to most lipid droplet associated proteins.

### EXAMPLE 3

### Establishment of Cell Lines Expressing PRP19 siRNA

(Step 1) Construction of PRP19 siRNA Expression Vector

In order to determine the nucleotide sequence of siRNA which can effectively downregulate PRP19 protein expression, the sequence of PRP19 siRNA was designed based on PRP19 mRNA sequence using an siRNA design program (http://www.ambion.com/techlib/misc/siRNA_finder.html) provided on the internet by Ambion company, to obtain the nucleotide sequence of SEQ ID NO: 1.

The pair of complementary oligonucleotides having SEQ ID NOs: 2 and 3 were designed as a template pair for the above designed siRNA in the custom synthesis conducted by Invitrogen company. The sense and antisense oligonucleotide sequences of the template were designed to comprise: 1) GATC and AGCT sequences on their 5′-ends, respectively, in order to insert the template between the BamHI and HindIII restriction sites of the commercially available siRNA expression vector, pSilencer 2.1-U6 puro vector (Ambion); and 2) a sequence containing not only the sense sequence for the designed siRNA, but also loop sequence and the antisense sequence for the designed siRNA, so as to express a double stranded siRNA having a hair-pin structure.

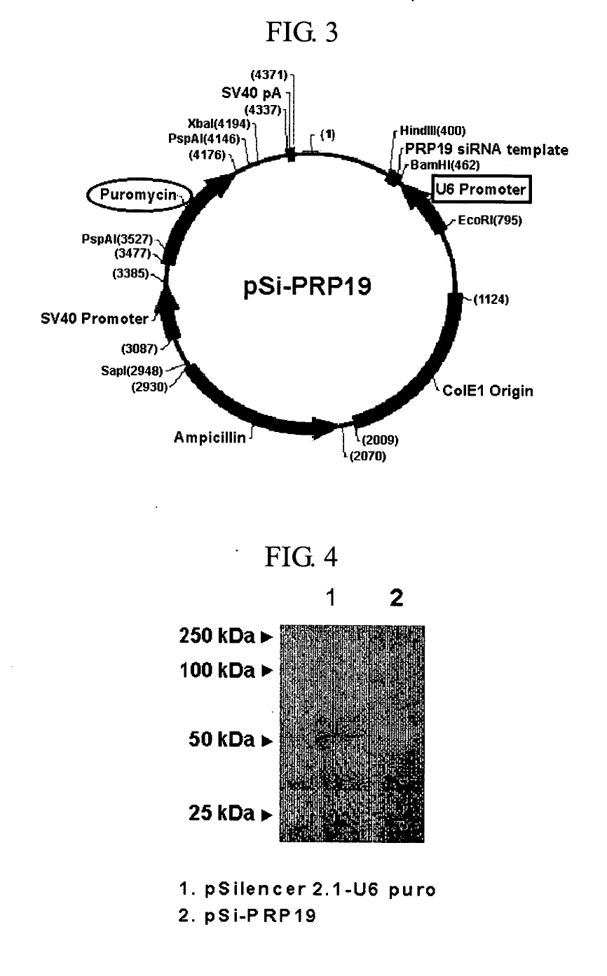

The complementary oligonucleotides thus synthesized were annealed with each other, and inserted in the ligation-ready pSilencer 2.1-U6 puro (Ambion) using T4 ligase, to obtain a PRP19 siRNA expression vector named pSi-PRP19. The cleavage map of vector pSi-PRP19 is shown in FIG. 3. The obtained vector was sequenced by the conventional DNA sequencing method to confirm that the construction of the desired vector was indeed achieved, which was transformed into E.coli to be cloned.

Step 2) Establishment of a Cell Line Expressing PRP19 siRNA

Some of the differentiated adipocytes obtained in Step 1) of Example 1 were transfected with pSi-PRP19 obtained in Step 1) using liposome (Lipofectamine, Invitrogen), and other cells were transfected with only pSilencer 2.1 -U6 puro vector as a control. The transfected cells were cultured in DMEM supplemented with 10% goat serum for 2 days, and then cultured in DMEM containing 3 μg/ml of puromycin for 10 days to select the transfected cell colonies. The selected colonies were subjected to a western blot analysis as descried in Step 2) of Example 1 to detect PRP19 protein expression. The result is shown in FIG. 4.

As shown in FIG. 4, PRP19 protein expression was downregulated in the cell line transfected with pSi-PRP 19 as compared with the control cell line.

### EXAMPLE 4

### The Effect of Downregulation of PRP19 Protein on Lipid Droplet Biogenesis

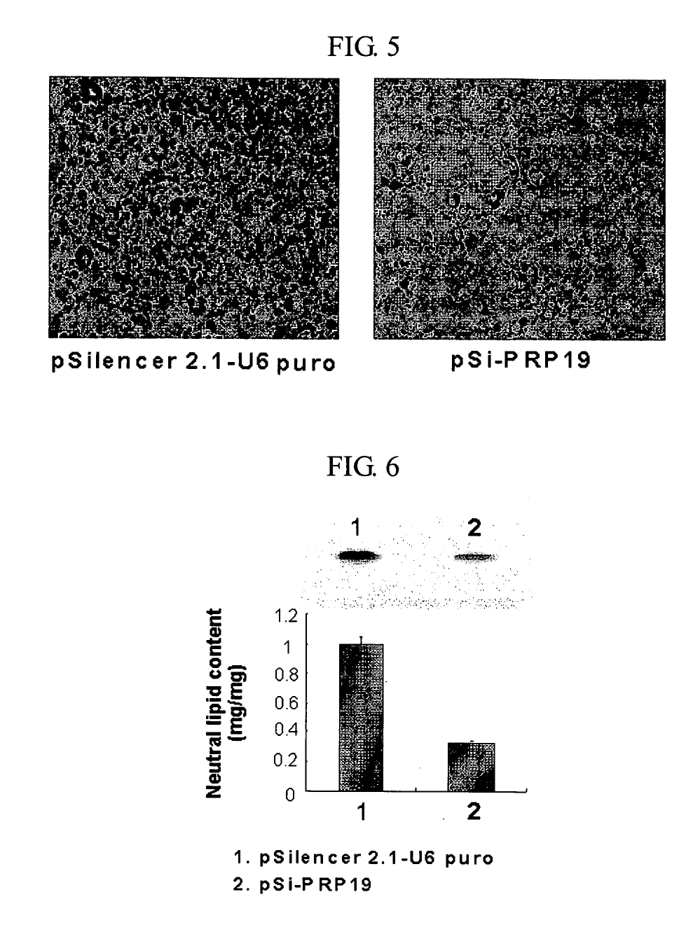

The cell line expressing PRP19 siRNA and the control cell line obtained in Example 3 were fixed with 3.7% formaldehyde and washed 3 times with PBS. Then, each of the fixed cell lines was stained with 0.2% oil red solution, washed with PBS for 15 min 4 times, and observed with camera microscopy (Axioplan 2, Carl Zeiss) to observe the lipid droplet biogenesis. The results are shown in FIGS. 5A and 5B.

The results show that the lipid droplet biogenesis was indeed suppressed significantly in the cell line expressing PRP19 siRNA relatively to the control cell line.

### EXAMPLE 5

### The Effect of Downregulation of PRP19 Protein on the Neutral Lipid Content

The cell line expressing PRP19 siRNA and the control cell line obtained in Example 3 were each cultured in 100 mm plates and harvested. Each of the harvested cell lines was mixed with 1 ml of a mixture of chloroform and methanol (2:1, v/v), and homogenized at room temperature for 15 min. The homogenized cell mixture was centrifuged at 1,000 rpm for 5 min to take a supernatant, and the supernatant was mixed with 0.2 volume of distilled water and centrifuged at 2,000 rpm for 5 min. The separated lower layer was sampled using a syringe, and the sample was diluted 10 folds with a mixture of chloroform and methanol (2:1, v/v) to measure the intracellular content of neutral lipids. The measurement was performed by applying each diluted sample on a HPTLC (high performance thin layer chromatography) plate using an automated sample applicator (LinomatIV, Camag), and developing the applied sample with automated multiple development chamber (AMD 2, Camag), according to the conventional method (L. Sek et al., J. Pharm. Biomed. Anal. (2001), 25, 651-661).

As shown in FIG. 6, the neutral lipid content of the cell line expressing PRP19 siRNA was reduced by over 70% in comparison with that of the control cell line.

### EXAMPLE 6

### The Effect of Downregulation of PRP19 Protein on the Expression of Lipogenic Proteins and Genes

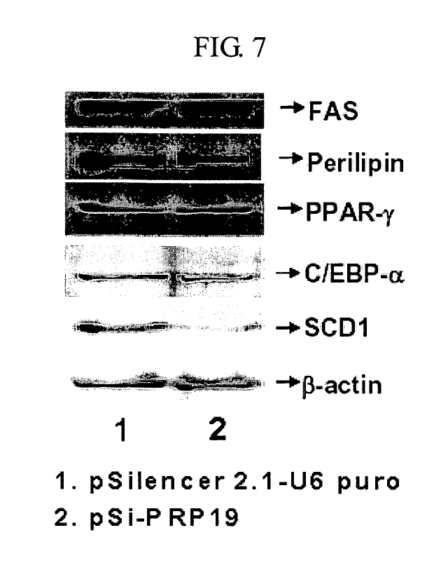

In order to detect the effect of downregulation of PRP19 protein on the expression of lipogenic proteins, the cell line expressing PRP19 siRNA and the control cell line obtained in Example 3 were each subjected to the western blot analysis as in Step 2) of Example 1, except for using anti-perilipin (a known lipid droplet-associated protein, Research Dioagnostics), FAS (fatty acid synthase, BD Biosciences), SCD-1 (a key rate-limiting enzyme in the synthesis of unsaturated fatty acids, Alpha Diagnostic International), PPAR-γ (an adipogenic transcription factor, Santa Cruz Biotechnology) or C/EBP-α (an adiopogenic transcription factor, Santa Cruz Biotechnology) monoclonal antibody as a primary antibody. The result is shown in FIG. 7.

As can be seen from the result in FIG. 7, the SCD-1 protein expression was much more suppressed in the cell line expressing PRP19 siRNA as compared to the control cell line, while no significant difference was found in the expressions of FAS, perilipin, PPAR-γ and C/EBP-α.

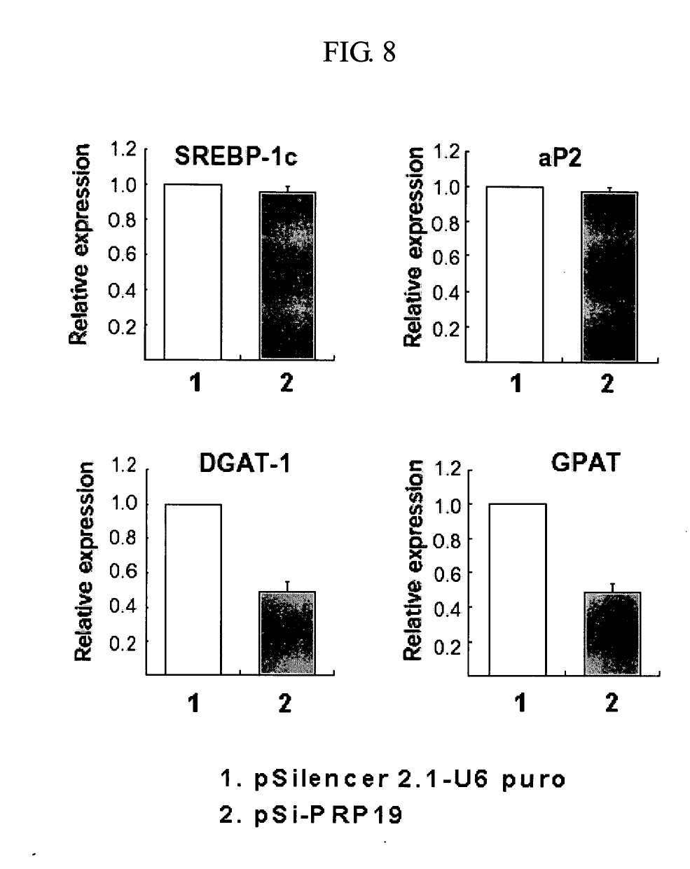

Further, the cell line expressing PRP19 siRNA and the control cell line obtained in Example 3 were each subjected to RT-PCR (50° C., 2 min; 95° C., 10 min; {95° C., 15 sec, 60° C., 1 min} 50 cycles) using commercially available primers (Applied Biosystems) designed to specifically amplify the genes of aP2 and SREBP-1c known as adipogenic transcription factors, and the genes of DGAT-1 and GPAT known as SCD-1 down-stream triacylglycerol synthesis enzymes, respectively. The result is shown in FIG. 8.

As shown in FIG. 8, the DGAT-1 and GPAT mRNA expressions were much more suppressed in the cell line expressing PRP19 siRNA as compared to the control cell line, while no significant difference was found in the mRNA expressions of aP2 and SREBP-1c.

These results demonstrate that the expression of SCD1, DGAT-1 and GPAT can be suppressed by downregulating the expression of PRP19 protein.

While the invention has been described with respect to the above specific embodiments, it should be recognized that various modifications and changes may be made to the invention by those skilled in the art which also fall within the scope of the invention as defined by the appended claims.

What is claimed is:

1. A pharmaceutical composition for the inhibition of lipogenesis comprising a regulator of PRP19 (precursor RNA processing 19) protein expression as an active ingredient and a pharmaceutically acceptable carrier.

2. The composition of claim 1, wherein the regulator is selected from the group consisting of: antisense RNAs, interfering RNAs (iRNAs) and small interfering RNAs (siRNAs) mediating RNA interference (RNAi) against the PRP19 gene expression, and the expression vectors thereof; transcription inhibitors of PRP19 gene; translation inhibitors of transcribed PRP19 mRNA; and inhibitors of PRP19 protein localization.

3. The composition of claim 2, wherein the regulator is siRNA mediating RNA interference (RNAi) against the PRP19 gene expression, or its expression vector.

4. The composition of claim 3, wherein the siRNA has the nucleotide sequence of SEQ ID NO: 1.

5. A method for inhibiting lipogenesis comprising the step of regulating the expression of PRP19 protein in a subject in need of the lipogenesis inhibition.

6. The method of claim 5, wherein the regulation of PRP19 protein expression causes the downregulation of the SCD1 (stearyl CoA desaturase-1), DGAT-1 (diacyglycerol acyltransferase-1) and GPAT (glycerol-phosphate acyltransferase) expression.

7. The method of claim 5, wherein the regulation of PRP19 protein expression is achieved by targeting PRP19 gene, targeting PRP19 mRNA, suppressing the transcription of PRP19 gene, suppressing the translation of PRP19 mRNA, or inhibiting the intracellular localization of PRP19 protein.

8. The method of claim 7, wherein the targeting of PRP19 mRNA is performed by using antisense RNA, iRNA or siRNA mediating RNA interference (RNAi) against the PRP19 gene expression, or an expression vector thereof.

9. The method of claim 5, wherein the subject is a mammal.

10. A method for screening a viable lipogenesis inhibitor comprising the steps of treating a subject with candidate inhibitors and analyzing PRP19 gene or RPR19 protein expression in the subject.

11. The method of claim 10, wherein the subject is selected from the group consisting of cell lines, tissues, and test animals which express PRP19 protein.

12. The method of claim 11, wherein the analysis of PRP19 gene or PRP19 protein expression is performed by using PRP19 gene or a fragment thereof, or an antibody specific for PRP19 protein.


SEQUENCE LISTING (3 sequences)

SEQ ID NO: 1 - RNA, 21 nt, Artificial Sequence
  siRNA for PRP19 protein
cagcucaucgacaucaaaguu

SEQ ID NO: 2 - DNA, 65 bp, Artificial Sequence
  sense template for siRNA of PRP19 protein
gatccgctcatcgacatcaaagttctcaagagaaactttgatgtcgatgagctgttttttggaaa

SEQ ID NO: 3 - DNA, 65 bp, Artificial Sequence
  antisense template for siRNA of PRP19 protein
agcttttccaaaaaacagctcatcgacatcaaagtttctcttgagaactttgatgtcgatgagcg